(19) United States
(12) Patent Application Publication
(10) Pub. No.: US 2004/0173533 A1
(43) Pub. Date: Sep. 9, 2004

(54) Separation of xylose and glucose

(76) Inventors: William A. Farone, Irvine, CA (US); Michael A. Fatigati, Rancho Santa Margarita, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/485,285

(22) PCT Filed: Jul. 24, 2002
(86) PCT No.: PCT/US02/23693

Related U.S. Application Data
(60) Provisional application No. 60/307,585, filed on Jul. 24, 2001.

(51) Int. Cl. B01D 15/08

(52) U.S. Cl. 210/656; 210/660; 127/46.2

(57) ABSTRACT

Disclosed is a process of chromatographically separating a material comprising a mixture of sugars, primarily xylose and glucose, into separate streams, one enriched in glucose and another enriched in xylose. In one preferred embodiment, the sugar mixture is obtained from the strong acid hydrolysis of cellulosic and hemicellulosic materials.

### RELATED APPLICATION INFORMATION

This application claims priority under 35 U.S.C. § 119(e) to provisional application serial No. 60/307,585, filed Jul. 24, 2001.

### BACKGROUND OF THE INVENTION

1. Field of the Invention

The present invention relates to a process of separating a material comprising a mixture of sugars, primarily xylose and glucose, into separate streams, one enriched in glucose and another enriched in xylose

2. Description of the Related Art

Mixtures of sugars are obtained from many sources. One source is from the treatment of biomass with concentrated acid to produce sugars, such as is detailed in U.S. Pat. Nos. 5,562,777, 5,597,714, and 5,726,046. In certain embodiments of these patented processes, cellulosic and hemicellulosic materials are treated with concentrated solutions of acid to produce a mixture of sugars comprising predominantly glucose and xylose. Oftentimes, the mixed sugars may be used substantially “as is” without the need for separating them prior to use. For example, in the aforementioned patents, it is noted that separation of the mixed sugar product is generally unnecessary when the sugars are to be used in fermentation processes. In some instances, however, it is desirable to separate the two major components of the sugar mixture from each other. Accordingly, there is a need for a suitable method of effecting the separation of glucose and xylose from a mixture comprising these sugars.

### SUMMARY OF THE INVENTION

In one embodiment, there is provided a method of separating a mixture of sugars primarily comprising glucose and xylose. The method comprises obtaining a mixture of sugars primarily comprising glucose and xylose in aqueous solution, feeding the mixture into a resin separation unit comprising one or more columns containing a resin capable of separating glucose and xylose thereby causing the separation of the mixture into a glucose stream comprising aqueous glucose and a xylose stream comprising aqueous xylose, and collecting the separate glucose and xylose streams wherein the xylose stream has a purity of at least 90%.

In another embodiment, there is provided a method of separating a mixture of sugars primarily comprising glucose and xylose. The method comprises obtaining a mixture of sugars primarily comprising glucose and xylose in aqueous solution, feeding the mixture into a resin separation unit comprising one or more columns containing a resin capable of separating glucose and xylose thereby causing the separation of the mixture into a glucose stream comprising aqueous glucose and a xylose stream comprising aqueous xylose having a purity of at least 90%, and collecting the separate glucose and xylose streams, wherein the one or more columns are styrene-divinylbenzene strong cation resin columns in which the functional group is sulfonate.

In another embodiment, there is provided a method of separating a mixture of sugars primarily comprising glucose and xylose. The method comprises obtaining a mixture of sugars primarily comprising glucose and xylose in aqueous solution, feeding said mixture into a resin separation unit comprising one or more columns containing DOWEX 99 resin (or another type of resin equivalent thereto) thereby causing the separation of the mixture into a glucose stream comprising aqueous glucose and a xylose stream comprising aqueous xylose having a purity of at least 90%; and collecting the separate glucose and xylose streams.

In preferred embodiments of the foregoing methods of separating a mixture of sugars, the mixture is obtained by a process comprising mixing cellulosic and/or hemicellulosic materials with a solution of about 25-90% acid by weight, thereby at least partially decrystallizing the materials and forming a gel that includes solid material and a liquid portion; diluting said gel to an add concentration of from about 20% to about 30% by weight and heating said gel, thereby at least partially hydrolyzing the cellulose and hemicellulose contained in said materials; separating said liquid portion from said solid material, thereby obtaining a mixed liquid containing sugars and acids; and separating the sugars from the acids in said mixed liquid by resin separation to produce a mixed sugar stream containing a total of at least about 15% sugar by weight, which is not more than 3% acid by weight. In a related process, the method of obtaining the mixed sugar further comprises mixing the separated solid material with a solution of about 25-90% sulfuric acid by weight thereby further decrystallizing the solid material to form a second gel that includes a second solid material and a second liquid portion; diluting said second gel to an acid concentration of from about 20% to about 30% by weight and heating said second gel to a temperature of about 80° to 100° C., thereby further hydrolyzing cellulose and hemicellulose remaining in said second gel; and separating said second liquid portion from said second solid material thereby obtaining a second liquid containing sugars and acid; and combining the first and second liquids to form a mixed liquid. In another related process, the method of obtaining the mixed sugar further comprises mixing the separated solid material with a solution of about 25-90% acid until the acid concentration of the gel is between about 20-30% acid by weight and heating the mixture to a temperature between about 80° C. and 100° C. thereby further hydrolyzing cellulose and hemicellulose remaining in said separated solid material and forming a second solid material and a second liquid portion; separating said second liquid portion from said second solid material thereby obtaining a second liquid containing sugars and acid; and combining the first and second liquids to form a mixed liquid. In preferred embodiments of the sugar production process described above, an acid separation is performed to separate the sugars from the majority of the acid. The acid separation comprises adding the mixed liquid to an acid resin separation unit comprising a cross linked polystyrene ion exchange resin bed, thereby producing a mixed sugar stream and an acid stream preferably containing less than 2% sugar.

### BRIEF DESCRIPTION OF THE DRAWINGS

FIGS. 1A through 1D illustrate pulse chromatograms of the type used to select suitable resins for use in separations according to a preferred embodiment.

FIG. 2 is a schematic view of the decrystallization and hydrolysis stages of a preferred method for producing a mixed sugar stream.

FIG. 3 is a schematic view of the fermentation and acid reconcentration stages of a preferred method for producing a mixed sugar stream.

### DETAILED DESCRIPTION OF THE PREFERRED EMBODIMENT

Introduction

Preferred embodiments provide a efficient process for separating mixtures of sugars comprising xylose and glucose. In especially preferred embodiments, a nearly pure xylose stream is obtained as a product. One source of such mixtures of sugar is from treating biomass containing cellulose and hemicellulose using concentrated acid, such as sulfuric, hydrochloric, hydrofluoric, or phosphoric acid. Preferred methods of obtaining a suitable mixed sugar stream are those set forth in U.S. Pat. Nos. 5,562,777, 5,597,714, and 5,726,046, the disclosures of which are hereby incorporated by reference in their entireties. Preferred processes disclosed in these patents are also set forth hereinbelow. Mixed sugar streams may, however, be obtained in ways other than those set forth in the foregoing patents, such that the extensive discussion of these patents herein should not be viewed as limiting upon the broader utility of the separation process.

The separation methods utilize chromatography to separate the sugars in the sugar stream. The term “stream” as used in combination with “sugar” or specific names of sugars refers to a composition comprising sugar (or the specific sugar named) and water; i.e. an aqueous sugar solution. In preferred embodiments, the sugar stream used as the startling product is in the range of about 40% to about 60% sugar by weight, including about 41%, 42%, 43%, 44%, 45%, 46%, 47%, 48%, 49%, 50%, 51%, 52%, 53%, 54%, 55%, 56%, 57%, 58%, and 59%. Use of starting sugar streams in this range (or slightly outside this range) maximize the separation and to reduce the total amount of resin needed to perform the separation. Sugar streams having higher concentrations of sugar, including 63%, 65%, 67%, 70% or more, and lower concentrations of sugar, including 43%, 40%, 37%, 35% or less, may also be used, although, as noted above, the efficiency may be reduced. A sugar stream starting material having a concentration of sugar outside the preferred range noted above may be used as is or it may be optionally concentrated (such as by evaporation of water) or diluted (such as by addition of water) to bring the concentration to a desired level. Similarly, solid sugars are preferably mixed with water to make a sugar stream of a desired concentration. Mixed sugar streams may also contain acid, such as is present following a strong acid hydrolysis process to produce a mixed sugar stream.

Any resin which is capable of performing the separation may be used. In one embodiment, the resin used for the separation is preferably an at least partially cross-linked styrene-divinylbenzene strong cation resin. In one embodiment, the functional group in the resin is sulfonate. Resins used in preferred methods preferably have one or more of the following characteristics: bead size of about 200-400 μm, about 300-350 μm, 320 μm and 350 μm (understanding that smaller beads mean a shorter length and higher pressure is needed); an exchange capacity of about 1-2 eq/L, including 1.5 eq/L; a water content of about 55-65, including 57-61; a particle density of about 1.2-1.3, including 1.28; and a tapped bed density of about 750-875 g/L, including about 785-849 g/L. In one especially preferred embodiment, the resin used is Dowex® 99 cation resins. These resins are available in both K⁺ and Ca²⁺ form from The Dow Chemical Company, Midland, Mich. Similar resins may also be suitable provided that they are able to achieve the necessary separation between the two sugars. However, not all resins of relatively similar chemical character have been found to be suitable in the present methods.

The chromatographic process proceeds by the sugars complexing with the cations present in the resin. The different sugars have different affinities or strengths of interaction with the cations such that a sugar having a weaker interaction or less affinity moves through the column faster than a sugar having a stronger interaction or more affinity for the cation. Sugars are known to complex with potassium, calcium, sodium and other metals, preferably Group I and II metals. Accordingly, resins containing these cations may be suitable for use in the methods herein.

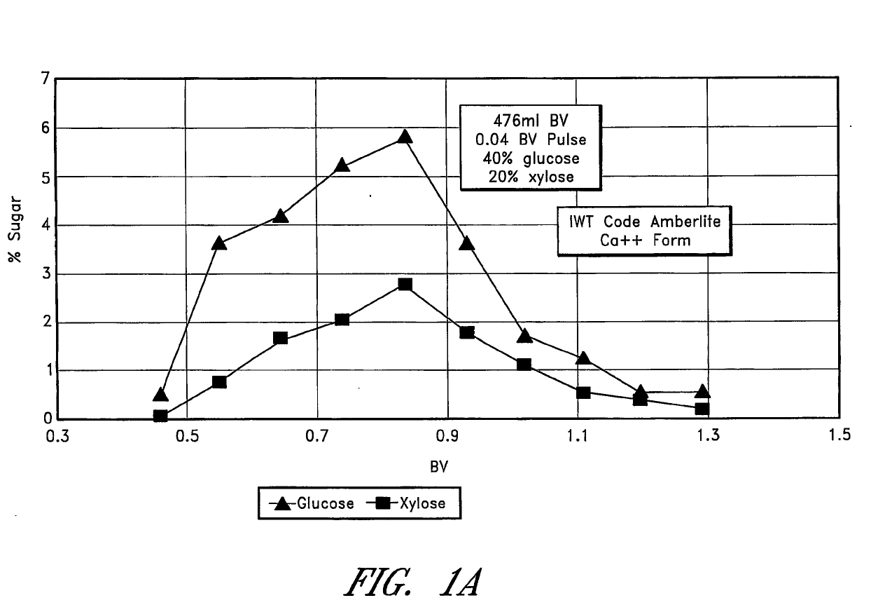

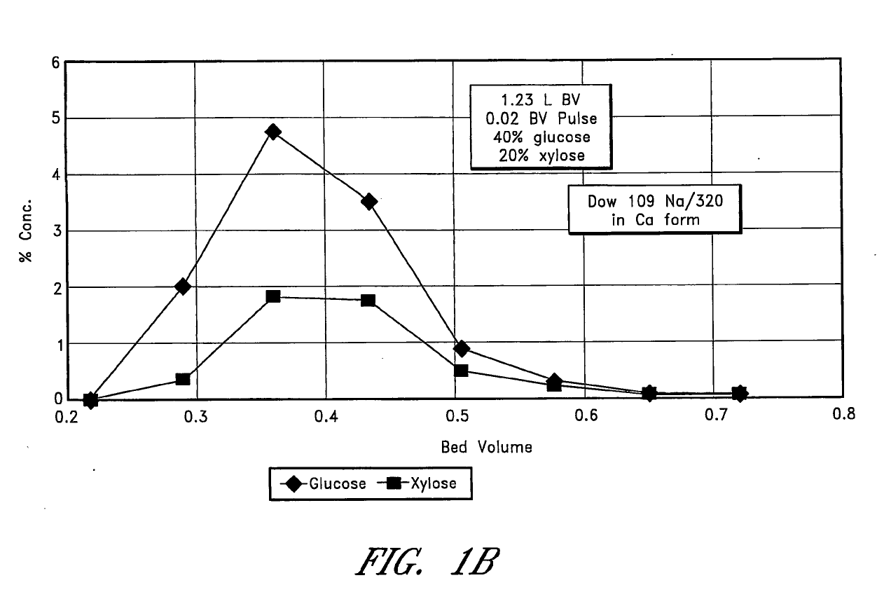

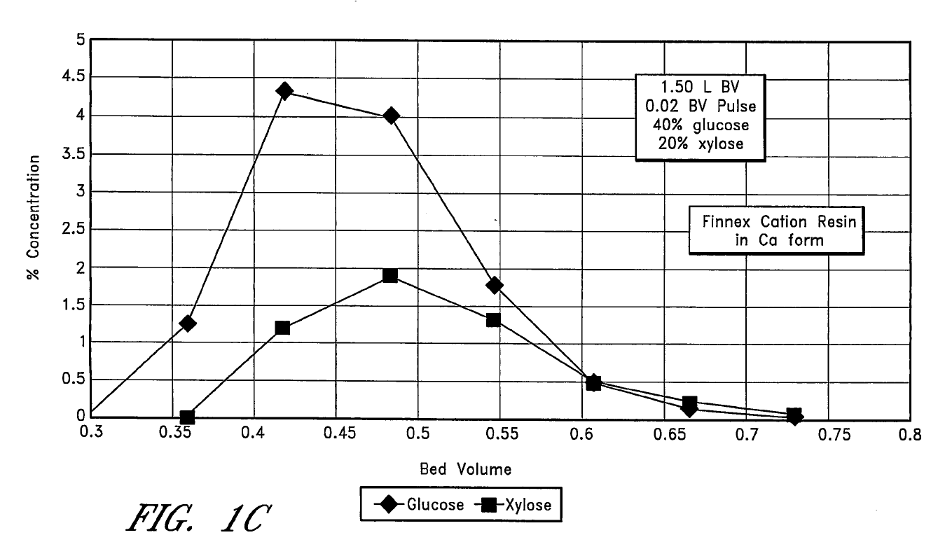

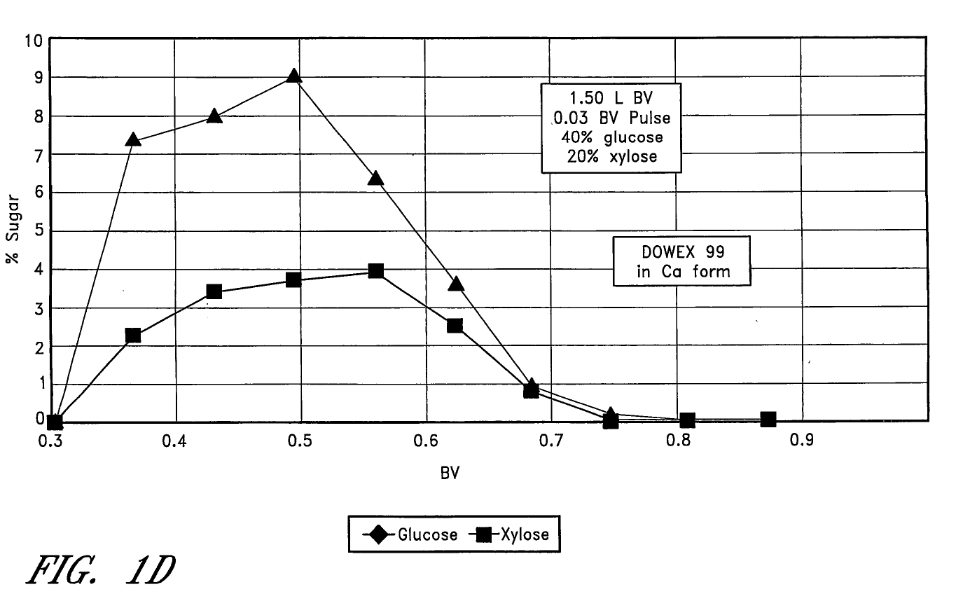

One preferred method of determining whether a resin is suitable is by running a test pulse chromatogram. The results of one set of test pulse chromatograms is shown in FIGS. 1A-D. In this test, a sugar stream was fed into the resin separation unit with water as the eluent using standard pulse chromatographic techniques. The composition of the stream emerging from the separation unit was analyzed and graphed as in FIGS. 1A-D. The results shown in FIGS. 1A-D are pulse chromatograms of four different resins, and were part of duplicate runs. The resins of FIGS. 1A-C (Amberlite Ca²⁺, Dow 109 Na/320 in Ca²⁺ form, and Finex cation resin in Ca²⁺ form, respectively did not work or did not work as well as the resin of FIG. 1D, the Dowex® 99 cation resin (Ca²⁺ form). The difference between the resin in 1D and the others is in the period when 0.5 to 0.55 bed volumes of liquid had been eluted. During this period, the concentration of glucose in the product stream coming out decreased by a significant rate while the xylose was still rising. This implies that there are conditions where the xylose adsorbed to the resin at a greater rate than the glucose, allowing for separation to occur. There is no comparable widely separated rate in any of the other pulse chromatograms. Although in FIG. 1C there is a period (between about 0.42 and 0.47 bed volumes) when the glucose is decreasing while the xylose is increasing, the difference in the rate between these two is not as great as that in FIG. 4D, such that separation would be much more difficult using the Finex cation resins. Accordingly, this method can be used to screen other resins for suitability in the present method. If similar results indicating a wide differentiation of adsorption between glucose and xylose under particular conditions are seen, that resin is very likely to be useful in the present method.

It was surprising to find that even in the case where the initial pulse chromatograms showed a great deal of overlap between the peaks for the xylose and glucose, excellent separations may be achieved for separation on a continuous basis based upon the information resulting from the pulse chromatograms when the above procedure is followed. It is within the abilities of one skilled in the art to determine a suitable separation apparatus, of any scale, based upon the information from the pulse chromatograms with minimal experimentation. Accordingly, even though the resin of FIG. 1D showed only a small difference in the pulse chromatogram for xylose and glucose, an excellent separation may still be achieved.

In preferred methods, the mixed sugar stream is continuously applied to the resin bed and eluted with about four to five times its weight with water. The columns in the resin separation unit are preferably heated or warmed to a temperature above room temperature, preferably from about 40° C. to about 70° C., including about 50° C., 55° C., and 60° C. The feed rates and flow rates are preferably chosen to maximize the separation of the glucose and xylose. In one embodiment, in which columns having a diameter of one inch are used, the feed rate for the mixed sugar stream is preferably about 1-5 ml/min, more preferably about 2-3 ml/min and the eluent feed rate is preferably about 5-20 ml/min, more preferably about 9-17 ml/min. Appropriate flow rates for columns of other sizes can be calculated with the understanding that the volume that is put into the columns increases proportional to the square of the radius.

As shown in the examples below, the separation process results in the production of two product streams from the mixed sugar stream, namely the xylose stream, which is enriched (up to 100%) in xylose, and a glucose stream which is enriched in glucose. These two product streams may also just be called the xylose stream and the glucose stream. Furthermore, xylose streams which are substantially free of glucose can be achieved following separation and up to 100% of the glucose can be recovered in the glucose stream when a pure xylose stream is desired. This allows processing of the xylose immediately after separation without any need for further purification.

In preferred methods, there are two materials going into the separation unit to start the process, namely the feed stream (i.e. the starting mixed sugar stream) and the eluent or desorbant, which is preferably aqueous material or water. As noted above, there are two output streams. The terms “recovery” and “purity” are used herein to describe the resulting xylose and glucose streams. When the mixed sugar stream is put into the resin essentially all of the glucose and xylose put in will come out. The purity of the glucose output stream is the percentage (by weight) of glucose in that stream compared to the total sugar present in that stream. The recovery of glucose in the glucose stream is the percentage (by weight) of glucose compared to the total amount of glucose available (i.e. the amount put into the resin separation unit). Xylose recovery and purity refer to the xylose in the xylose stream. Thus purity measures the amount of desired product in the product stream and recovery measures how much of the total amount of desired product is in that stream.

In preferred embodiments, the purity of the xylose stream is greater than about 90%, more preferably greater than about 95%, including about 100%; the xylose recovery is preferably greater than about 60%, more preferably greater than about 75%, including about 80%; the purity of the glucose stream is preferably greater than about 60%, more preferably greater than about 75%; and the glucose recovery is preferably greater than about 85%, more preferably greater than about 90%, including about 95%, and about 100%.

These examples were done using a 20 column pseudo-moving bed chromatographic unit from U.S. Filter. The resin used was Dowex 99 (Ca²⁺, 350 micron) in columns measuring 1 inch in diameter by 27 inches in length. Results obtained from this type of equipment are routinely scaled to any desired commercial size.

Furthermore, in the examples which follow, all values are averaged over 24 hours or longer. There is always a significant amount of material in the columns of the separation unit at any given time. This “hold-up” of material makes a mass balance around the columns for purposes of demonstrating the effectiveness and efficiency of the methods in the examples difficult because the material that is put in during any 24 hour period is not exactly the material that comes out. When the test period for the following examples starts, the columns are already full and the columns are still full when the test period ends.

Table 1 below provides some dimensional analysis of the test equipment used for the examples which follow under one set of operating conditions. These conditions were chosen to match pump flows and are given only to show the translation of flow rates in various units measured over either 1 or 3 days. Accordingly, these are values for one embodiment, and are not to be taken as necessary for the proper operation of the inventive method.

TABLE 1
Relation of Flow Dimensions

| | Flow rate | Flow rate | Flow rate | Flow rate | Flow rate |
|---|---|---|---|---|---|
| Stream | (ml/min) | (L/day) | (gal/day) | (lbs/day) | (gal/3 days) |
| Feed | 2.5 | 3.6 | 0.9 | 7.96 | 2.7 |
| Water | 6.5 | 9.36 | 2.34 | 20.69 | 7.02 |
| glucose | 6.5 | 9.36 | 2.34 | 20.69 | 7.02 |
| xylose | 2.5 | 3.6 | 0.9 | 7.96 | 2.7 |

The hold-up of liquid is about 2.2 liters. l a day, the total flow is 12.7 liters. Thus the error in mass balance due to the hold-p can be as high as 17% if the system is not in steady state. For this reason, the unit is run for at least 24 hours at a set of operating conditions before the 24 hour period for which the analysis is made. In steady state operation, the average value of the flows will be constant over long periods of time.

A flow of constant composition is not necessary to allow the system to operate properly. Fairly wide fluctuations in input values can be tolerated while still achieving good separations. It is necessary, however, to make accurate mass balance measurements that are discussed in the examples. The data in all of the examples represents averages over a 24-hour period of operation.

### EXAMPLE 1

TABLE 2

| | Flow rate | Glucose | Xylose | Purity | Recovery |
|---|---|---|---|---|---|
| Stream | (ml/min) | (wt. %) | (wt. %) | (%) | (%) |
| Feed | 3.0 | 19.04 | 38.90 | | |
| Water | 16.36 | | | | |
| glucose | 9.21 | 6.50 | 2.10 | 75.96 | 88.3 |
| xylose | 11.75 | 0.66 | 10.24 | 93.9 | 86.4 |

A neutralized hydrolysate stream was concentrated to 19.04% glucose and 38.90% xylose. The solution density was 1.26 g/cc. This solution entered the chromatographic unit at a flow rate of 3.0 ml/min and the system was held at 60° C. The elution water flow rate was 16.36 ml/min. The two product streams left the unit at 9.21 ml/min for the glucose rich stream (density 1.03 g/cc) and 11.75 ml/min for the xylose rich stream (density 1.05 g/cc). The xylose stream purity was 93.9% and the xylose recovery in this stream was 86.4%. The glucose stream purity was 75.96% and the glucose recovery in this stream was 88.3%.

### EXAMPLE 2

TABLE 3

| | Flow rate | Glucose | Xylose | Purity | Recovery |
|---|---|---|---|---|---|
| Stream | (ml/min) | (wt. %) | (wt. %) | (%) | (%) |
| Feed | 2.85 | 14.94 | 26.49 | | |
| Water | 14.08 | | | | |
| glucose | 13.62 | 4.75 | 0.89 | 84.2 | 100.0 |
| xylose | 3.21 | 0.0 | 6.97 | 100.0 | 64.6 |

A neutralized hydrolysate stream was concentrated to 14.94% glucose and 26.49% xylose. The solution density was 1.20 g/cc. This solution entered the chromatographic unit at a flow rate of 2.85 ml/min and the system was held at 60° C. The elution water flow rate was 14.08 ml/min. The two product streams left the unit at 13.62 ml/min for the glucose rich stream (density 1.03 g/cc) and 3.21 ml/min for the xylose rich stream (density 1.03 g/cc). The xylose stream purity was 100.0% and the recovery of xylose in this stream was 64.6%. The glucose stream purity was 84.2% and the glucose recovery in this stream was 100.0%.

### EXAMPLE 3

TABLE 4

| | Flow rate | Glucose | Xylose | Purity | Recovery |
|---|---|---|---|---|---|
| Stream | (ml/min) | (wt. %) | (wt. %) | (%) | (%) |
| Feed | 2.97 | 12.91 | 31.29 | | |
| Water | 14.16 | | | | |
| glucose | 12.67 | 2.92 | 1.73 | 62.8 | 100.0 |
| xylose | 4.19 | 0.0 | 18.86 | 100.0 | 78.9 |

A mixed glucose-xylose stream of 12.91% glucose and 31.29% xylose was prepared and fed into the chromatographic unit. The solution density was 1.22 g/cc. This solution entered the chromatographic unit at a flow rate of 2.97 ml/min and the system was held at 60° C. The elution water flow rate was 14.16 ml/min. The two product streams left the unit at 12.67 ml/min for the glucose rich stream (density 1.03 g/cc) and 4.19 ml/min for the xylose rich stream (density 1.08 g/cc). The xylose stream purity was 100.0% and the recovery of xylose in this stream was 78.9%. The glucose stream purity was 62.8% and the glucose recovery in this stream was 100.0%.

### EXAMPLE 4

TABLE 5

| | Flow rate | Glucose | Xylose | Purity | Recovery |
|---|---|---|---|---|---|
| Stream | (ml/min) | (wt. %) | (wt. %) | (%) | (%) |
| Feed | 3.08 | 18.26 | 32.90 | | |
| Water | 9.08 | | | | |
| glucose | 13.22 | 4.36 | 1.76 | 71.2 | 98.0 |
| xylose | 3.81 | 0.29 | 25.39 | 98.9 | 81.6 |

A mixed glucose-xylose stream of 18.26% glucose and 32.90% xylose was prepared and fed into the chromatographic unit. The solution density was 1.22 g/cc. This solution entered the chromatographic unit at a flow rate of 3.08 ml/min and the system was held at 60° C. The elution water flow rate was 9.08 ml/min. The two product streams left the unit at 13.22 ml/min for the glucose rich stream (density 1.03 g/cc) and 3.81 ml/min for the xylose rich stream (density 1.10 g/cc). The xylose stream purity was 98.9% and the recovery of xylose in this stream was 81.6%. The glucose stream purity was 71.2% and the glucose recovery in this stream was 98.0%.

### EXAMPLE 5

TABLE 6

| | Flow rate | Glucose | Xylose | Purity | Recovery |
|---|---|---|---|---|---|
| Stream | (ml/min) | (wt. %) | (wt. %) | (%) | (%) |
| Feed | 2.52 | 19.5 | 41.26 | | |
| Water | 11.86 | | | | |
| glucose | 11.40 | 5.95 | 1.68 | 78.0 | 93.1 |
| xylose | 4.11 | 1.13 | 26.30 | 95.9 | 86.0 |

A mixed glucose-xylose stream of 19.50% glucose and 41.26% xylose was prepared and fed into the chromatographic unit. The solution density was 1.27 g/cc. This solution entered the chromatographic unit at a flow rate of 2.52 ml/min and the system was held at 60° C. The elution water flow rate was 11.86 ml/min. The two product streams left the unit at 11.40 ml/min for the glucose rich stream (density 1.02 g/cc) and 4.11 ml/min for the xylose rich stream (density 1.11 g/cc). The xylose stream purity was 95.9% and the recovery of xylose in this stream was 86.0%. The glucose stream purity was 78.0% and the glucose recovery in this stream was 93.1%.

Preferred Processes for Producing the Mixed Sugar Stream

What follows is a description of preferred processes for making the mixed sugar stream. In the following subsections, the processes or methods referred to are those for producing the mixed sugar stream. The processes produce a sugar stream that is rich in xylose and glucose with small amounts of galactose, arabinose and mannose also being made in most cases. When the starting material is rice straw, waste paper, wood, sugar cane bagasse, corn stalks and various grasses, xylose and glucose generally comprise about 98% or more of the sugars produced.

Decrystallization

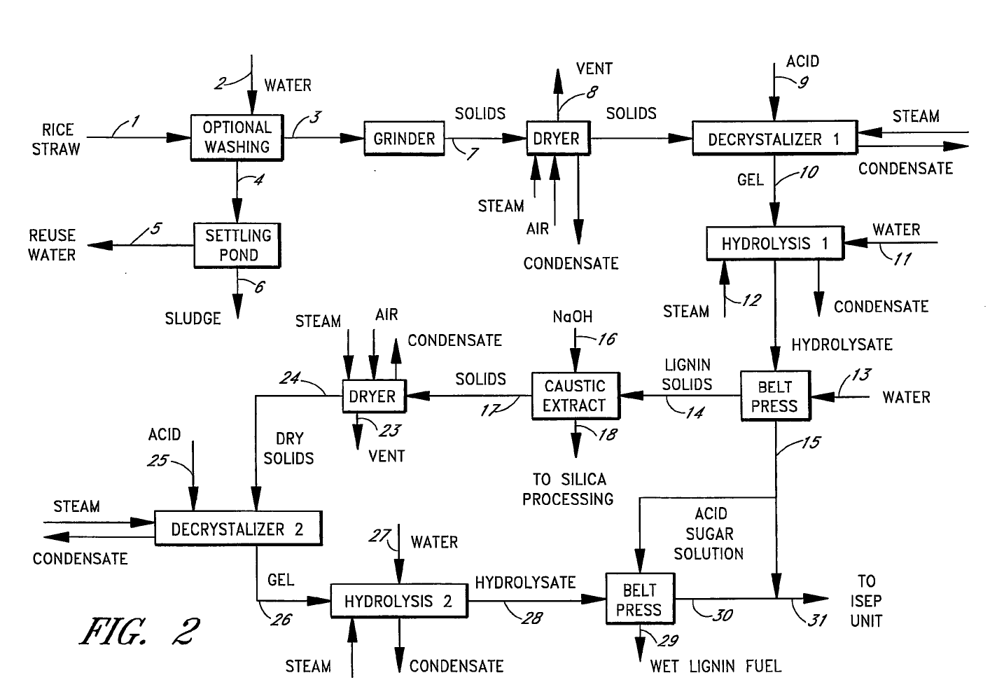

The raw materials used in preferred methods are blended such that the cellulose and hemicellulose content is at least 65%, and more preferably about 75%. As an optional first step in the process, the biomass can be washed to remove gross dirt and contamination. As seen in FIG. 2, the rice straw 1, the biomass used as an example throughout the figures, is washed with water 2. In many instances, washing of the biomass is not necessary, as most “dirt” (clay, sand, small pieces of rocks) will pass through the process unchanged and end up in the lignin cake. Advantageously, the method can be used with a variety of raw materials, including rice straw, which, because of its high silica content, is more difficult to process than other materials. It should be noted, however, that the principles of this method of making sugars are not limited to any particular type of biomass, but are intended to apply to a broad range of materials. Rice straw is intended to be merely exemplary in nature.

After the washing is complete, the used water is preferably transferred to a settling pond 4, to allow dirt and other sediment to collect on the bottom 6, after which the water can be reused 5 to wash the next portion of rice straw before processing.

Once the rice straw has been cleaned, it may be optionally dried 8, preferably to a moisture content of approximately 10%. After drying, the material is ground 7 into particles. For dense materials, that is, materials such as wood and rice straw having a density of greater than about 0.3 gm/cc, the particles preferably range in size from about 0.075 mm to 7 mm. Preferably, the particles range in size from 3 mm to 7 mm, and are of an average size of 5 mm. For materials having a density less than about 0.3 gm/cc, such as paper, particle size can be increased up to about 25 mm, with a preferred average size of 15 mm. It should be noted that for some materials the order of the drying and grinding steps should be reversed. That is, the material may be wet ground using a device such as a hydropulper and then dried.

The rice straw is now ready for the decrystallization stage. In this process, raw materials containing cellulose and/or hemicellulose are first mixed with concentrated acid 9 at a concentration of between 25% and 90% to effect decrystallization. Preferably, the concentration of acid used is between 70% and 77%. Preferably, the acid used is sulfuric acid, but other acids including hydrochloric, hydrofluoric, and phosphoric acid may also be used. To reduce the occurrence of metal attack on the reaction chamber by the concentrated acid used, some of the biomass is placed in the reactor first, followed by the acid solution, followed by the gradual addition of the rest of the biomass. In addition, the reactor is preferably lined with thin layers of polytetrafluoroethylene (PTFE, known commercially as TEFLON), polyvinylidene (PVDF, known commercially as KYNAR), or a copolymer of chlorotrifluoroethylene (CTFE) and ethylene (known commercially as HALAR). High density polyethylene, polyvinyl chloride, and polypropylene can also be used.

The acid should be added to achieve a ratio of the weight of pure acid to the weight of cellulosic and hemicellulosic materials of at least 1:1. Preferably, the ratio achieved is 1.25:1. The addition of acid to the biomass results in the formation of a thick gel 10, having a viscosity of approximately 1.5 to 2 million cp, which is thoroughly mixed prior to hydrolysation. Advantageously, this mixture of the raw material with the acid results in the disruption of the bonds between the cellulose and hemicellulose chains, making the long chain cellulose available for hydrolysis.

The decrystallization is preferably performed such that the temperature does not exceed 80° C., and is preferably in the range of 60-80° C., or more preferably, the decrystallization should be below 60° C. with optimum results obtained when the cake is kept below a temperature of 35-40° C. If the temperature during decrystallization exceeds 80° C., much of the C₅ sugars will be lost in the subsequent hydrolysis. The preferred sugar production method uses conditions which conserve the more reactive sugars that are produced earlier in the hydrolysis process. The decrystallization step prevents premature hydrolysis and consequently increased degradation of the sugars.

In the decrystallization step, the heat generated when large quantities of biomass and acid are mixed cannot be readily removed by conduction due to the low conductivity of the cake mixture. The removal under vacuum of water from the mixture, however, is generally sufficient to cool the mixture. The addition rate of the biomass, and thus the rate of the entire decrystallization process, is directly proportional to the rate at which water can be removed by the vacuum pump. The removal of water from the system by vacuum does not require the addition of solvent to remove the heat via evaporation, and the water, along with the small amount of acid entrained in the water, can be added back to the system after condensation, thus maintaining precise composition control and eliminating any waste product.

As the size of the reactor increases, the surface to volume ratio decreases. Since the decrystallization and hydrolysis material has very low thermal conductivity, the vacuum system removes an increasing percentage of the heat as the size of the reactor increases. In experiments performed in glass lined vessels the vacuum removed almost all of the heat due to the further decrease in the thermal conductivity of the glass. The vacuum also reacts much more rapidly than heat transfer through a surface.

The decrystallization stage is further described in Examples 6-8 which follow.

### EXAMPLE 6

Rice straw, containing 75% by weight of cellulose plus hemicellulose, and weighing 50.01 grams was mixed with 66.82 grams of 77% H₂SO₄. The rice straw was slowly added to the H₂SO₄ such that there was excess liquid available after each increment was added. The temperature was kept below 80° C. After the last amount of rice straw was added the resulting gelatinous mass was thoroughly mixed.

### EXAMPLE 7

Rice straw weighing 50.04 grams was mixed with 98.91 grams of 77% H₂SO₄. A small portion of the rice straw was placed in the reactor, the acid solution was added, and the remaining rice straw was slowly added to the H₂SO₄ such that there was excess liquid available after each increment was added. The temperature was kept below 80° C. by removing the water present in the mixture under vacuum. An initial pressure of 275 mm Hg (a vacuum of 757.26) was used to vaporize the solution at 40° C. Pressure of 180 mmHg (vacuum of 580 mm Hg) was sufficient to keep the solution cool at 40° C. After the last amount of rice straw was added the resulting mass was thoroughly mixed.

### EXAMPLE 8

A mixture of wood prunings and newspaper weighing 100.00 grams was mixed with 167.63 grams of 77% H₂SO₄. The wood prunings were ground to 3-7 mm in size and 40 grams were mixed with 60 grams of the newspaper which had been shredded into approximately 6 mm pieces. The mixture was slowly added to the H₂SO₄ such that there was excess liquid available after each increment was added. The temperature was kept below 80° C. After the last amount of prunings and newspaper was added the resulting gelatinous mixture was thoroughly mixed.

First Hydrolysis

After the decrystallization stage, the concentrated acid in the mixture is diluted, preferably to a concentration of between 20% and 30%, and preferably using recycled water 11. This reduces the viscosity of the mixture from about 1.5 to 2 million cp to about 400,000 cp. The mixture is then heated to a temperature of between 80° and 100° Celsius and continuously mixed to effect hydrolysis 12. Mixing at low rotations per minute (rpm) is preferred, approximately 10-30 rpm. A second mixer at higher rpm is useful to keep the material in the vicinity of the slow speed mixer.

The hydrolysis is allowed to continue for between 40 and 480 minutes, depending on the temperature and the concentration of cellulose and hemicellulose in the raw materials. If the proper time is exceeded, the rate of degradation of the hexoses and pentoses will exceed their rate of formation. Thus, to increase the sugar yield, one may stop the first hydrolysis after a time and remove the sugars, and then perform a second hydrolysis to convert the remainder of the cellulose and hemicellulose to sugars. After hydrolysis, the acid sugar solution is separated from the remaining solids, preferably by pressing 15, filtering, or filter pressing.

The filterability of the hydrolysate slurry is affected by the temperature at which the decrystallization takes place. The cooler the decrystallization can be kept the easier it is to filter the subsequent hydrolysis product. The decrystallization should be below 60° C. with optimum results obtained when the cake was kept below a temperature of 35-40° C. It is generally impractical to keep it any cooler as the viscosity increases and the vacuum required to cool the mixture is too costly to maintain.

The benefit to filterability and higher yields from lower decrystallization temperatures indicates that the reactor design must be able to turn over the reacting materials and expose them to the lower pressure such that there is preferably less than a 6° C. difference in temperature anywhere in the reactor. Multiple blade mixer designs are better suited to this than single blades.

Another way to enhance separability of the solids remaining after decrystallization and hydrolysis is to make sure the lignin present in the biomass is adequate to allow a filter press to be used to remove the sugar-acid solution after hydrolysis. If there is insufficient lignin present in the biomass and all of the cellulose and hemicellulose gets converted into sugars, the solution will be very difficult to filter press. If the biomass were all cellulose and hemicellulose there would be no need to filter press as the sugar acid solution could go directly to the acid-sugar separation unit. However, whenever some of the biomass is not simply cellulose and hemicellulose it is desirable to have lignin present to act as an aid to filtering. In addition, the presence of lignin in the biomass also provides the following advantages: (1) it serves as a material upon which to deposit other materials such as inorganic materials and oxidized sugars; and (2) it acts as a coproduct which can provide fuel value or be used as a media for growing plants or as a topsoil additive.

It has been found that a combination of biomass materials with an average value of at least 5% lignin (dry basis) are preferred to assure enough cake is present to allow filtration. Lignin amounts of 7% may be more preferable as a compromise between filtration and optimal product yields. Higher amounts of lignin in the biomass make filtration even easier but the amount of sugars produced will be reduced because the additional lignin composition means less cellulose and hemicellulose will be available for hydrolysis.

The hydrolysis stage is further described in Examples 9-11 below.

### EXAMPLE 9

To the resulting gelatinous mass from Example 6, 54.67 grams of water were added for hydrolysis to reduce the acid concentration of the total mixture to 30%. The sample was heated to 100° C. for 60 minutes. Some water evaporation occurred during the heating. The gelatinous mass was pressed to yield 93 grams of a liquid which was 17.1% sugars and 35.52% acid.

### EXAMPLE 10

After the resulting gelatinous mass in Example 7 was thoroughly mixed, 104.56 grams of water were added to reduce the acid concentration of the total mixture to 30%. The sample was heated to 100° C. for 60 minutes. The gelatinous mass was pressed to yield 188.9 grams of a liquid which was 16.5% sugars and 34.23% acid.

### EXAMPLE 11

After the resulting gelatinous mass from Example 8 had been thoroughly mixed, 162.62 grams of water were added for hydrolysis to reduce the acid concentration of the total mixture to 30%. The sample was heated to 100° C. for 60 minutes. Some water evaporation occurred during the heating. The gelatinous mass was pressed to yield 214.3 grams of a liquid which was 17.6% sugars and 36.85% acid.

After pressing, the resulting cake containing the solid matter was washed with 170 grams of water and pressed again to yield a liquid which was 16.3% acid and 8.92% sugar, which was used for subsequent washing to increase the sugar yield.

Second Decrystallization and Hydrolysis

To increase the sugar yields, an optional second decrystallization and a second hydrolysis step may be undertaken. The second decrystallization step is unnecessary in most instances, however, for bulky materials such as wood, a second decrystallization step may be performed when the first decrystallization step fails to adequately decrystallize the cellulosic and hemicellulosic materials.

The solids remaining after the first hydrolysis or any subsequent processing after the first hydrolysis are dried 23. The dry solids 24 are mixed with concentrated sulfuric acid 25 at a concentration of between 25% and 90% to effect the second decrystallization, if necessary. Preferably, the acid concentration is between 70% and 77%. It is not necessary to hold the material for the same length of time as in the first decrystallization. In fact, this second decrystallization can be as short as the few minutes it takes to mix the acid and the solids. This second decrystallization also results in the formation of a thick gel 26.

The concentrated acid is then diluted, preferably to a concentration of between 20% and 30% and preferably using recycled water 27. The mixture is then heated to effect a second hydrolysis. Alternatively, in those cases where a second decrystallization is unnecessary, the solids remaining after the first hydrolysis or any subsequent treatment, are treated with 20-30% acid and heated to effect a second hydrolysis. The resulting gel 28 is pressed or filtered to obtain a second acid sugar stream 30, and the streams from the two hydrolysis steps are combined. The remaining lignin-rich solids are collected and optionally pelletized for fuel 29, or used as feedstock. Advantageously, pelletization of the lignin-rich cake helps reduce the waste produced by the process of the present invention.

Protein-type materials can be included as part of agricultural or waste materials used as feedstocks to the process of the present invention. Although sulfuric acid has been used to analyze for protein and amino acid nitrogen by releasing the nitrogen as ammonia (the so-called Kjeldahl test), there is no indication of ammonia release from protein in grasses and other plant materials in this process. This allows the use of products with protein without losing the nitrogen. The protein and amino acid nitrogen is still available, for example, as a natural nitrogen fertilizer, when the cake remaining after hydrolysation is used as a soil amendment. In addition, the nitrogen provides additional value to the lignin-rich cake as an animal food supplement.

The second decrystallization and hydrolysis steps are further explained in Examples 12 and 13 which follow.

### EXAMPLE 12

The cake formed from pressing after the first hydrolysis of rice straw was collected and dried to a moisture content of 10%. The cake, containing 41% cellulose and weighing 50.03 grams, was mixed with 33.28 grams of 77% H₂SO₄ to achieve a ratio of pure acid to cellulose of 1.25 to 1. The cake was slowly added to the acid and mixed until a thick gel was formed. The resulting pure acid concentration in the mixture was 30.75%, thus 17.00 grams of water was added to provide a final pure acid concentration of 25.5%. The mixture was then heated at 100° C. for 50 minutes. After cooling, the gel was pressed to recover 31.45 grams of a liquid containing 18.2% sugar and 21.1% acid. The cake containing the solids remaining after pressing was washed with 25 grams of water to produce a solution which was 15.4% sugar and 19.7% acid.

The pressed cake was dried to a water content of about 10%. This cake was shown to have a fuel value of 8,600 BTU per pound. This fuel material, which is primarily lignin with unrecovered sugar, some sugar degradation products, and some unreacted cellulose burned extremely well but left an ash that contained about 7% silica.

### EXAMPLE 13

A rice straw hydrolysis cake which had been treated to remove silica and weighed 500 grams was mixed with 77% H₂SO₄ to achieve a ratio of pure acid to cellulose of 1.25 to 1. The cake was slowly added to the acid and mixed until a thick gel was formed. Water was then added to provide a final pure acid concentration of 25.5%. The mixture was then heated at 100° C. for 50 minutes. After cooling, the gel was pressed to recover a liquid containing both sugar and acid. The cake containing the solids remaining after pressing was washed with water to produce a second solution containing sugar and acid.

The pressed cake was dried to a water content of about 10%. This cake was shown to have a fuel value of 8,600 BTU per pound. This fuel material, which is primarily lignin with unrecovered sugar, some sugar degradation products, and some unreacted cellulose burned extremely well and left an ash with a silica content of <1%.

Separation of Acid and Sugar

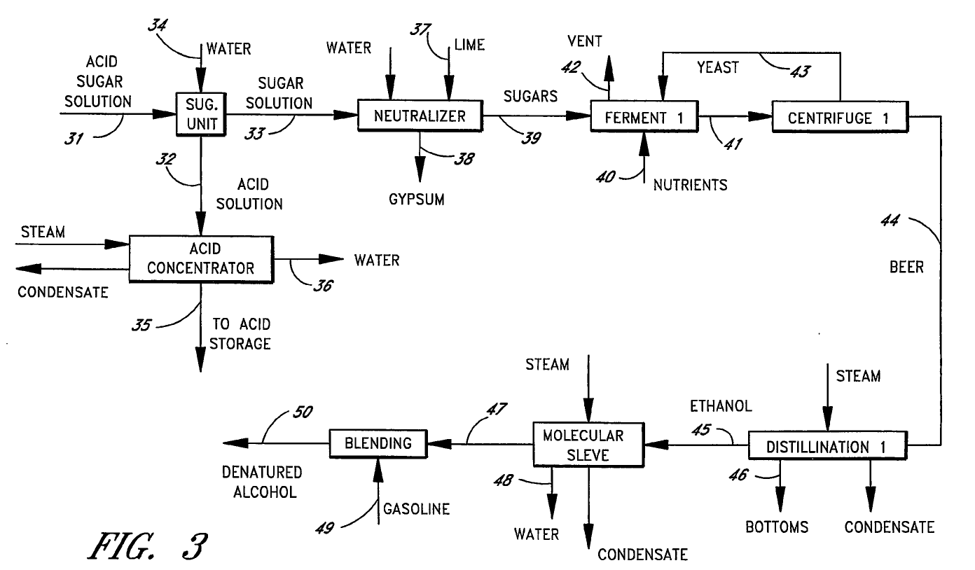

This preferred method of producing a mixed sugar stream also provides for an improved method for separating the acid and sugar in the hydrolysate produced from the acid hydrolysis of cellulosic and hemicellulosic material or from any mixture of sugars containing a strong acid. Referring now to FIG. 3, the acid sugar stream 31 is processed through a separation unit, which comprises either cationic or anionic resin for the separation of the acid and sugars. In one embodiment, a strong acid polystyrene-divinylbenzene resin bed is used. The resin is preferably cross-linked with divinylbenzene, which is preferably at a concentration of between 6% and 8%, and treated with sulfuric acid such that it has a strong acid capacity of at least 2 meq/g. Several such resins are commercially available, including DOWEX 40166, available from Dow Chemical, Finex GS-16, available from Finex, Finland, Purolite PCR-771, available from Purolite Inc., Bala Cynwyd Pa., and IR-118, available from Rohm and Haas. In a particularly preferred embodiment, the resin used is DOW XFS 43281.01, available from Dow Chemical. The resin is preferably in the form of beads which are between 200 to 500 micrometers in diameter. The flow rate of the resin bed is preferably 2 to 5 meters per hour, and the bed preferably has a tapped bed density of between 0.6 and 0.9 g/ml. The resin bed should be heated, preferably to a temperature of between 40-60° C. Higher temperatures can be used, but will result in premature degradation of the resin bed. Lower temperatures will result in separations which are not as effective.

In the case of a cationic resin, the sugar is adsorbed on the resin as the acid solution moves through 32. Once the acid has eluted, the resin may optionally be purged with a gas which is substantially free of oxygen, preferably less than 0.1 ppm dissolved oxygen. This gas acts to push any remaining acid out of the resin, resulting in a cleaner separation.

After the elution of the acid stream, the resin is washed with water 34 that is substantially free of oxygen. The dissolved oxygen content of the water is preferably below 0.5 ppm, and more preferably, below 0.1 ppm. This washing results in the production of a sugar stream 33 containing at least 98% of the sugars in the hydrolysate that was added to the separation unit.

As a result of the separation process, three streams are collected: the acid stream, the sugar stream, and a mixed acid-sugar stream which is recycled through a second separation process. The add stream 32 is reconcentrated and recycled for reuse, as will be explained more fully below. The sugar stream 33, preferably contains at least 15% sugar and not more than 3% acid. The purity of the sugar can be calculated as a percentage of the nonaqueous components of the sugar stream.

The inclusion of acid concentration as high as 3% in the sugar stream does not generally cause problems for further processing. However, loss of significant proportions of sugar with the acid upon separation can decrease the overall economy of the process.

In an exemplary, ideal separation process, 100 grams of water would be used to elute a 100 gram sample solution containing 30 grams of acid, 15 grams of sugar, and 55 grams of water from a separation column. In the case of perfect separation, the sugar stream would contain 15 grams of sugar and 85 grams of water. This would leave 30 grams of acid and 70 grams (100+55−85) of water for recovery of acid in the same concentration, 30%, as the original solution.

However, a typical elution for the 100 gram sample solution referred to above would require that about 200 grams of water be added to the column. The sugar stream is still 15%, but now the acid stream contains 170 grams (200+55−85) of water and 30 grams of acid, resulting in a 15% acid concentration. Thus, if the acid stream was 95% pure with an acid concentration of 15%, approximately 1.5 grams of sugar would be lost with the acid with every elution. If the sugar stream was 95% pure at a 15% concentration, only 0.75 grams of acid would be lost with every elution. Thus, because the acid stream contains twice as much material as the sugar stream, for achieving the best separation between acid and sugar, the purity of the acid stream is a more important factor than the purity of the sugar stream.

Similar techniques may be used when the resin used for separating acid and sugar is an anionic resin. The separation of the acid and sugars using cationic resins is further explained in Examples 14-21 which follow.

### EXAMPLE 14

An acid sugar stream produced by the hydrolysis of cellulosic and hemicellulosic material was separated by flowing it through a 50 cm diameter glass column of 1.2 liters volume packed with PCR-771, a strong acid cation exchange resin available from Purolite, Inc. The column was held at 60° C. and the volumetric flow rate was 70 ml/min, which translates into a linear flow rate of about 0.8 meters per hour. Three streams were collected, the acid stream, the sugar stream and a mixed stream for recycle to another resin bed. The acid stream was 96.8% pure (sum of acid and water). The sugar stream was 86.8% pure (sum of sugar and water). Overall, the recovery of the acid was 97.3% and the recovery of the sugar was 95.5%.

### EXAMPLE 15

A portion of hydrolysate liquid produced by the acid hydrolysis of cellulosic and hemicellulosic material was separated by flowing it through a 50 cm diameter glass column of 1.2 liters volume packed with PCR-771, a strong acid cation exchange resin available from Purolite, Inc. The column was held at 40° C. and the volumetric flow rate was 70 ml/min. Three streams were collected, the acid stream, the sugar stream and a mixed stream for recycle to another resin bed. The add stream was 95.1% pure (sum of acid and water). The sugar stream was 93.1% pure (sum of sugar and water). Overall, the recovery of the acid was 98.6% and the recovery of the sugar was 90.6%.

### EXAMPLE 16

A hydrolysis liquid containing 34.23% H₂SO₄ and 16.5% sugar was separated by flowing it through a 50 cm glass column of 1.2 liters volume packed with PCR-771, a strong acid cation exchange resin available from Purolite, Inc. The column was held at 60° C. and the volumetric flow rate was 70 ml/min. Three streams were collected, the acid stream, the sugar stream and a mixed stream for recycle to another resin bed. The acid stream was 96.47% pure (sum of acid and water). The sugar stream was 92.73% pure (sum of sugar and water). Overall, the recovery of the acid was 97.9% and the recovery of the sugar was 95.0%.

### EXAMPLE 17

Hydrolysate liquid produced from the hydrolysis of newspaper was found to contain 31.56% acid and 22.97% sugar. The liquid was separated by flowing it through a 50 cm glass column of 1.2 liters volume packed with PCR-771, a strong acid cation exchange resin available from Purolite, Inc. The column was held at 40° C. and the volumetric flow rate was 70 ml/min. Three streams were collected, the acid stream, the sugar stream and a mixed stream for recycle to another resin bed. The acid stream was 96.7% pure (sum of acid and water). The sugar stream was 90.9% pure (sum of sugar and water). Overall, the recovery was 99.5% for the acid and 96.7% for the sugar.

### EXAMPLE 18

Hydrolysate liquid produced from the hydrolysis of newspaper was found to contain 31.56% acid and 22.97% sugar. A portion of the liquid was separated by flowing it through a 50 cm glass column of 1.2 liters volume packed with Finex GS-16, a strong acid cation exchange resin available from Finex, Finland. The column was held at 60° C. and the volumetric flow rate was 70 ml/min. A second portion of the liquid was also separated by flowing it through a 50 cm glass column of 1.2 liters volume packed with Finex GS-16. This column was held at 40° C. and the volumetric flow rate was 70 ml/min. In both cases, three streams were collected, the acid stream, the sugar stream and a mixed stream for recycle to another resin bed. The acid streams were at least 90% pure (sum of acid and water). The sugar streams were at least 94% pure (sum of sugar and water).

### EXAMPLE 19

A hydrolysate containing 15% sugar and 30% add was separated using a 50 cm glass column of 1.2 liters volume packed with DOW XFS 43281.01 resin, available from Dow Chemical. The column was held at 60° C. and the volumetric flow rate was 65 ml/min. After adding the hydrolysate, the column was eluted with boiled and cooled distilled water. The acid stream was 97.0% pure, and the sugar stream was 97.2% pure. The amount of swelling between the acid and water phases on the resin was 2.48%.

A second addition of the same hydrolysate to the column followed by elution recovered essentially all of the acid and sugar, with over 99.1% recovery, and 97.2% sugar purity and 92.3% acid purity. The elution rate during the separation was 65 ml/min.

### EXAMPLE 20

An AST LC1000 rotating resin bed device manufactured by Advanced Separation Technologies, Inc. was used to separate the sugar-acid mixtures. The device consisted of 20 columns of resin, each column containing 2 liters of bed volume. The columns were filled with Finex GS-16 resin held at 60° C. In one run of 8 hours, the feed consisted of 14.89% sugar and 23.79% acid. The elution rate was 244 ml/min, which corresponds to linear rate of 0.12 m/min or 7.3 m/hour. The sugar product purity was 94.6% and the acid product purity was 92.4%. The sugar recovery was 84% with a concentration of 13.9%. The acid recovery was 97.5% with a concentration of 7.5%.

### EXAMPLE 21

An AST LC1000 rotating resin bed device manufactured by Advanced Separation Technologies, Inc. with a total bed volume of 15.2 liters was used to separate the sugar-acid mixtures. The columns were filled with Purolite PCR-771. The feed contained 12.6% sugar and 18.9% acid. The elution flow rate was 117 ml/min. The sugar purity in the recovered stream was 92.4% and the acid purity was 92.1% when the columns were operated at 60° C.

Upon analysis of the sugar mixture, it was determined that the distribution of sugars produced by the process of the present invention is remarkably consistent and consists primarily of 5 single C5 or C6 sugars; there is no evidence of dimer, trimer or other short chain polymeric sugars produced using this procedure. In addition, there is evidence, obtained, e.g., by gas chromatography of the trimethyl allyl esters, of the presence of xylitol in the C5 sugars. Xylitol is a reduced form of xylose and is easier for microorganisms to utilize.

Concentration and Recycling of Acid

The acid solution 32 recovered from the separation unit can be concentrated and recycled for reuse in the earlier stages of the process of the present invention. Concentration of the acid up to 35% is achieved through the use of a standard single stage evaporator 36. A triple effect evaporator, such as that available from Chemetics (Toronto, Ontario, Canada), is preferably used, resulting in increased concentrations of 70-77%. The water 35 recovered in the concentrator can be used as elution water in the resin separator unit. Similar equipment may also be used to concentrate the sugar stream prior to separation.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments. Similarly, the various features and steps discussed above, as well as other known equivalents for each such feature or step, can be mixed and matched by one of ordinary skill in this art to perform methods in accordance with principles described herein.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein, but instead by reference to claims attached hereto.

What is claimed is:

1. A method of separating a mixture of sugars primarily comprising glucose and xylose, comprising:
obtaining a mixture of sugars primarily comprising glucose and xylose in aqueous solution;
feeding said mixture into a resin separation unit comprising one or more columns containing a resin capable of separating glucose and xylose thereby causing the separation of the mixture into a glucose stream comprising aqueous glucose and a xylose stream comprising aqueous xylose; and
collecting the separate glucose and xylose streams;
wherein said xylose stream has a purity of at least 90%.

2. A method according to claim 1, wherein the one or more columns are styrene-divinylbenzene strong cation resin columns.

3. A method according to claim 1 or 2, wherein the columns have sulfonate functional groups.

4. A method of separating a mixture of sugars primarily comprising glucose and xylose, comprising:
obtaining a mixture of sugars primarily comprising glucose and xylose in aqueous solution;
feeding said mixture into a resin separation unit comprising one or more columns containing a resin capable of separating glucose and xylose thereby causing the separation of the mixture into a glucose stream comprising aqueous glucose and a xylose stream comprising aqueous xylose having a purity of at least 90%; and
collecting the separate glucose and xylose streams;
wherein said one or more columns are partially cross-linked styrene-divinylbenzene strong cation resin columns in which the functional group is sulfonate.

5. A method of separating a mixture of sugars primarily comprising glucose and xylose, comprising:
obtaining a mixture of sugars primarily comprising glucose and xylose in aqueous solution;
feeding said mixture into a resin separation unit comprising one or more columns containing DOWEX 99 resin or the functional equivalent thereof, thereby causing the separation of the mixture into a glucose stream comprising aqueous glucose and a xylose stream comprising aqueous xylose having a purity of at least 90%; and
collecting the separate glucose and xylose streams.

6. A method according to any of the preceding claims, wherein the one or more columns are in Ca²⁺ form.

7. A method according to any of the preceding claims, wherein the one or more columns have an exchange capacity of about 1.5 eq/L.

8. A method according to any of the preceding claims, wherein the one or more columns have a resin in the form of beads about 200-400 μm in diameter.

9. A method according to any of the preceding claims, wherein the one or more columns have a resin in the form of beads about 300-350 μm in diameter.

10. A method according to any of the preceding claims, wherein the one or more columns have a tapped bed density of about 785-849 g/L.

11. A method according to any of the preceding claims, wherein the purity of the xylose stream is at least 95%.

12. A method according to any of the preceding claims, wherein the xylose stream is substantially free of glucose.

13. A method according to any of the preceding claims, wherein the glucose recovery is at least 85%.

14. A method according to any of the preceding claims, wherein the glucose recovery is at least 95%.

15. A method according to any of the preceding claims, wherein the mixture of sugars comprises 40%-60% sugars by weight.

16. A method according to any of the preceding claims, further comprising concentrating the mixture of sugars prior to feeding it into the one or more columns.

17. A method of separating a mixture of sugars primarily comprising glucose and xylose, according to any of the preceding claims, wherein said mixture is obtained by a process comprising:
mixing cellulosic and/or hemicellulosic materials with a solution of about 25-90% acid by weight, thereby at least partially decrystallizing the materials and forming a gel that includes solid material and a liquid portion;
diluting said gel to an acid concentration of from about 20% to about 30% by weight and heating said gel, thereby at least partially hydrolyzing the cellulose and hemicellulose contained in said materials; and
separating said liquid portion from said solid material, thereby obtaining a mixed stream containing sugars and acids.

18. The method according to claim 17, further comprising separating the sugars from the acids in said mixed stream by resin separation to produce a mixed sugar stream containing a total of at least about 15% sugar by weight, which is not more than 3% acid by weight.

19. The method according to claim 18, wherein the separation comprises:
adding the mixed liquid to an acid resin separation unit comprising a cross linked polystyrene ion exchange resin bed, thereby producing a mixed sugar stream and an acid stream containing less than 2% sugar.

20. The method according to claim 17, 18 or 19, further comprising the following steps after the separating liquid from solid step:
mixing the separated solid material with a solution of about 25-90% sulfuric acid by weight thereby further decrystallizing the solid material to form a second gel that includes a second solid material and a second liquid portion;
diluting said second gel to an acid concentration of from about 20% to about 30% by weight and heating said second gel to a temperature of about 80° to 100° C., thereby further hydrolyzing cellulose and hemicellulose remaining in said second gel; and
separating said second liquid portion from said second solid material thereby obtaining a second liquid containing sugars and acid; and
combining the first and second liquids to form a mixed liquid.

21. The method according to claim 17, 18 or 19, further comprising the following steps after the separating liquid from solid step:
mixing the separated solid material with a solution of about 25-90% acid until the acid concentration of the gel is between about 20-30% acid by weight and heating the mixture to a temperature between about 80° C. and 100° C. thereby further hydrolyzing cellulose and hemicellulose remaining in said separated solid material and forming a second solid material and a second liquid portion;
separating said second liquid portion from said second solid material thereby obtaining a second liquid containing sugars and acid; and
combining the first and second liquids to form a mixed liquid.

22. The method according to any of the preceding claims, further comprising washing said materials containing cellulose and hemicellulose.

23. The method according to any of the preceding claims, further comprising drying said materials containing cellulose and hemicellulose.

24. The method according to any of the preceding claims, wherein the acid is selected from the group consisting of hydrochloric acid, hydrofluoric acid and phosphoric acid.

25. The method according to any of the preceding claims, wherein the acid is sulfuric acid.

26. The method according to any of the preceding claims, wherein the heating is performed for between 40 and 480 minutes.

27. The method according to any of the preceding claims, wherein the heating is performed at a temperature of 100° C. for 40-110 minutes.

28. The method according to any of the preceding claims, wherein the heating is performed at a temperature of 90° C. for 80-220 minutes.

29. The method according to any of the preceding claims, wherein the heating is performed at a temperature of 80° C. for 160 to 480 minutes.

30. The method according to any of the preceding claims, wherein the hydrolysis is performed at atmospheric pressure.

31. The method according to any of the preceding claims, wherein the acid used to effect decrystallization is at a concentration of from about 70% to about 77% by weight.

32. The method according to any of the preceding claims, wherein the acid solution is added to achieve a ratio of pure acid to cellulosic and hemicellulosic material of at least about 1:1.

33. The method according to any of the preceding claims, wherein the acid solution is added to achieve a ratio of pure acid to cellulosic and hemicellulosic material of about 1.25:1.

34. The method according to any of the preceding claims, wherein the decrystallizing of the materials is performed at a temperature of less than 80° C.

35. The method according to any of the preceding claims, wherein the decrystallizing of the materials is performed at a temperature of less than 60° C.

36. The method according to any of the preceding claims, wherein the decrystallization step further comprises removal of heat using a vacuum to remove water which is recycled to the decrystallization step.

37. The method according to any of the preceding claims, wherein the raw materials contain from about 50% to about 85% cellulose and hemicellulose.

38. The method according to any of the preceding claims, wherein the separation is performed using a resin separation unit wherein the sugars are adsorbed on a strong acid resin.

39. The method according to any of the preceding claims, wherein the resin is cross linked with divinylbenzene and treated with sulfuric acid to produce a strong acid resin.

40. The method according to any of the preceding claims, wherein the divinylbenzene is at a concentration of from about 6% to about 8%.

41. The method according to any of the preceding claims, wherein the resin is in the form of beads having a diameter of from about 200 to about 500 micrometers.

42. The method according to any of the preceding claims, wherein liquid flows through the resin bed with an average linear flow rate of from about 2 to about 5 meters per hour.

43. The method according to any of the preceding claims, further comprising heating said resin bed to a temperature of from about 40 to about 60 degrees Celsius.
